(19) United States
(12) Patent Application Publication
(10) Pub. No.: US 2024/0282459 A1
(43) Pub. Date: Aug. 22, 2024

(54) MACHINE LEARNING BASED DECISION SUPPORT SYSTEM FOR SPINAL CORD STIMULATION LONG TERM RESPONSE

(71) Applicant: Albany Medical College, Albany, NY (US)

(72) Inventors: Julie G. Pilitsis, Albany, NY (US); Amir Hadanny Hadanny, Albany, NY (US)

(73) Assignee: Albany Medical College, Albany, NY (US)

(21) Appl. No.: 18/566,695

(22) PCT Filed: Jun. 4, 2021
(86) PCT No.: PCT/US2021/035941

§ 371 Date: Dec. 4, 2023

(51) Int. Cl. G16H 50/50; A61N 1/36; G16H 10/20; G16H 10/60; G16H 50/70

(52) U.S. Cl. CPC G16H 50/50; A61N 1/36062; G16H 10/20; G16H 10/60; G16H 50/70

(57) ABSTRACT

A system for predicting a spinal cord stimulation having a user interface for entry of features from a new patient and a machine learning engine having a cluster stage trained to evaluate the plurality of patient features to identify a cluster corresponding to the plurality of features of the patient from a plurality of clusters and a prediction stage trained to output a patient predicted outcome based a predictive model corresponding to the identified cluster. The plurality of features may comprise patient demographics, pain descriptors, pain questionnaire data, psychiatric comorbidities, spinal imaging, activity, medications, non-psychiatric comorbidities, and past spinal cord stimulation results.

### BACKGROUND OF THE INVENTION

### 1. Field of the Invention

The present invention relates to medical treatment decision making tools and, more specifically, an approach for assessing the potential outcome of spinal cord stimulation (SCS) for a particular patient.

### 2. Description of the Related Art

Neuromodulation treatment including approaches such spinal cord stimulation (SCS), which is an FDA-approved treatment for managing chronic pain, most commonly for medically refractory back and neck pain and complex regional pain syndrome. The devices have been increasingly used over the last 5 years at a growth rate of 20%, in part due to the opioid epidemic. Despite patients undergoing psychological assessment and a trial of SCS prior to implant, suboptimal outcomes after SCS implant may occur in as many as 50% of patients at 2 years. Though these numbers have improved recently with the advent of new waveforms, explant rates hover around 10% and failure rates are estimated at 25-30%. There remains a lack of a clear understanding of which patients benefit long term. Thus, the ability to accurately predict patients who will not benefit from SCS would reduce the high financial burden of failed implants that plague the neuromodulation field. Moreover, this would provide an objective datapoint to augment the clinician's decision about when to pursue alternate therapies in lieu of SCS. Currently, patient selection for SCS is based on the subjective experience of the implanting physician. As provider experience is often less reliable than evidence-based care, it is essential to determine which variables have the greatest influence on patient outcomes so algorithms may be established.

In pain, ML has been used to identify radiographic and electrophysiological biomarkers of chronic pain and to define the phenotypes of patients with chronic lumbar radiculopathy for predictive purposes. ML has also demonstrated the ability to predict positive treatment response in specific subtypes of chronic pain patients. Alexander Jr et al. demonstrated that the combination of two ML methods can classify patients' response to pregabalin. Azimi et al. used neural network algorithm to predict patients' satisfaction following lumbar stenosis surgery with high accuracy (96%). Use of ML in SCS, however, has been limited. De Jaeger suggested a predictive model using logistic regression and regression trees (CART) in patients who had failed standard SCS and responded to a salvage SCS waveform. Although predictive features were identified, the model was not validated internally or externally. Recently, Goudman et al. used ML algorithms to predict high frequency (HF)-SCS responders with 50% pain relief, but with limited accuracy and predictive performance. Accordingly, there is need in the art for an approach that can predict patent responses to spinal cord stimulation treatment.

### BRIEF SUMMARY OF THE INVENTION

The present invention comprises an approach that uses machine learning (ML) modeling to predict patient response to spinal cord stimulation treatment. The approach of the present invention demonstrated that at least two distinct clusters of patients exist and that each cluster's long-term response with spinal cord stimulation can be predicted with 70-75% success. Given the significant healthcare costs of poor response to chronic pain therapies, the ML approach of the present invention provides for high predictive performance as a decision support tool in patient selection that can contribute to more effective pain management. The present invention was demonstrated by applying a combination of unsupervised clustering and supervised classification to obtain individualized models for each subgroup/cluster of patients in the largest single-center database of prospectively collected longitudinal SCS outcomes.

In a first embodiment, a system for predicting a spinal cord stimulation according to the present invention has a user interface configured to accept data representing a plurality of features from a new patient for whom a prediction of spinal cord stimulation is desired. The system also has a machine learning engine having a cluster stage trained to evaluate the plurality of patient features to identify a cluster corresponding to the plurality of features of the patient from a plurality of clusters and a prediction stage trained to output a patient predicted outcome based a predictive model corresponding to the identified cluster, referred to as classification. The plurality of clusters of the cluster stage are defined according to K-means clustering of data representing the plurality of features from patients having known outcomes. The predictive model comprises a machine learning algorithm trained with data representing the plurality of features from patients having known outcomes. The machine learning algorithm may comprise logistic regression, random forest, XGBoost, elasticnet, support vector machine, Naïve Bayes, or combinations thereof. The plurality of features may comprise patient demographics, pain descriptors, pain questionnaire data, psychiatric comorbidities, spinal imaging, activity, medications, non-psychiatric comorbidities, and past spinal cord stimulation results.

In another embodiment, the present invention comprises a method for predicting a spinal cord stimulation outcome. In a first step, the method comprises the step of collecting a plurality of patient features from a patient whose spinal cord stimulation outcome is to be predicted. In another step, the method comprises using a machine learning engine having a cluster stage trained to evaluate the plurality of patient features to identify a cluster corresponding to the plurality of features of the patient from a plurality of clusters. In another step, the method comprises using the identified cluster to evaluate the patient data with a prediction stage of the machine learning engine trained to output a patient predicted outcome based a predictive model that corresponds to the identified cluster.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a system for predicting the outcome of spinal cord stimulation in a prospective patient according to the present invention:

FIG. 2 is diagram of a graphic user interface for a system for predicting the outcome of spinal cord stimulation according to the present invention:

FIG. 3 is a schematic of a machine learning engine for a system for predicting the outcome of spinal cord stimulation according to the present invention:

FIG. 4 is a flowchart of the training of the machine learning engine using historical patient data according to the present invention.

FIG. 5 is a graph of the distortions as a function of number of clusters shows a pseudo-elbow of the graph at K=3 clusters:

FIG. 6 is a graph of the entire data set projected on the two principal components:

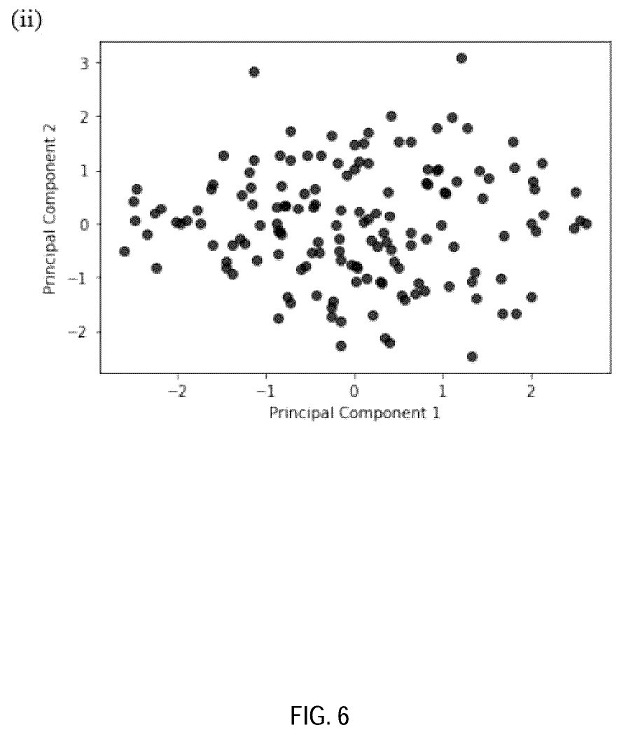

FIG. 7 is a graph of K=3 clustering of the data projected on the two principal components demonstrating less separation between the clusters and that the third cluster includes a lower number of patients:

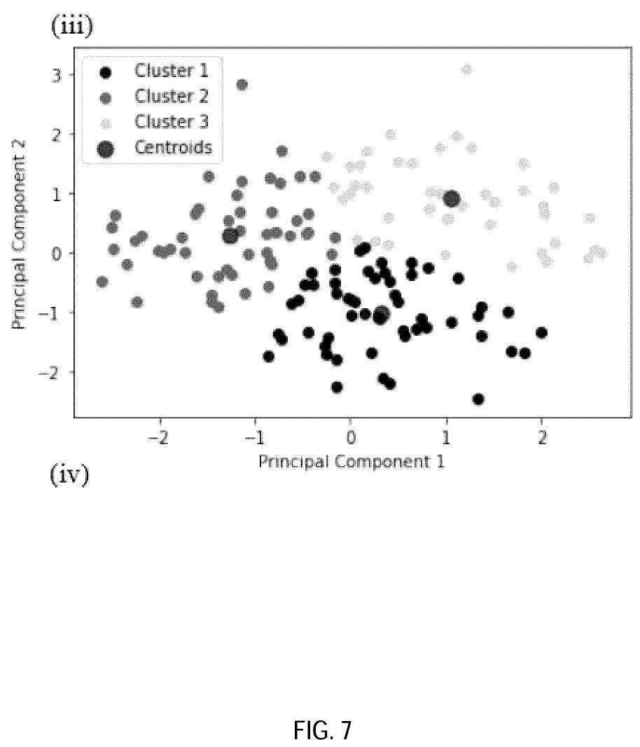

FIG. 8 is a graph of K=2 clustering of the data projected on the two principal components demonstrates two distinct clusters (cluster 1 data points in black and cluster 2 data point in grey);

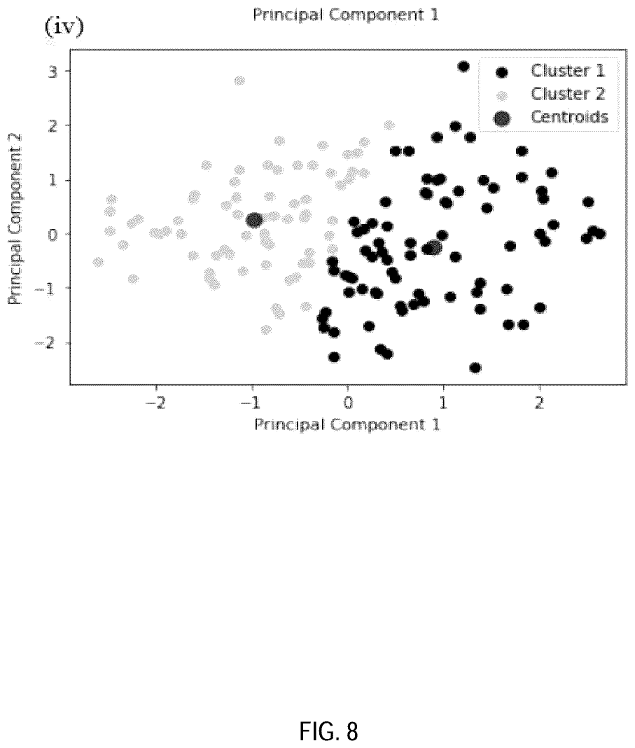

FIG. 9 is a flowchart of patient actions used to evaluate the approach of the present invention:

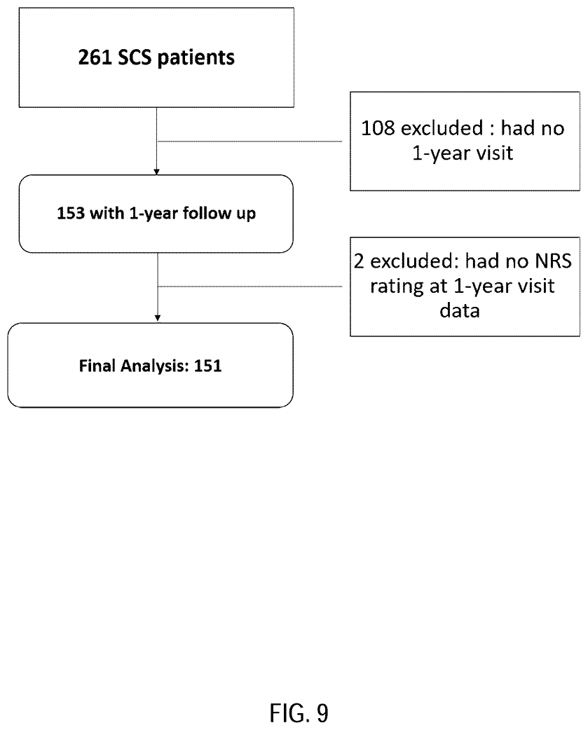

FIG. 10 is a graph of ROC curves comparison for LR on cluster 1, LR on cluster 2 and LR, RF, XGBoost on entire cohort;

FIG. 11 is a graph of models mean AUCs compared; and

FIG. 12 is a features correlation heatmap where features with significant multicollinearity (correlation>0.7) were excluded.

### DETAILED DESCRIPTION OF THE INVENTION

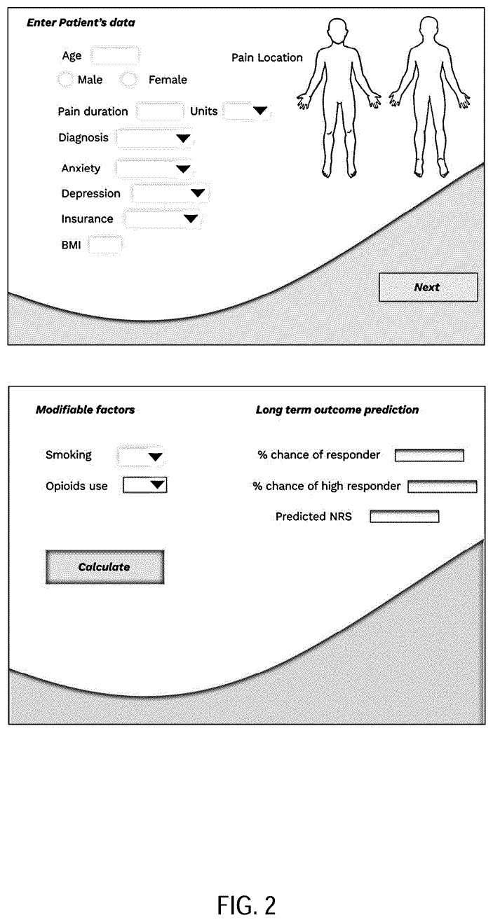

Referring to the figures, wherein like numerals refer to like parts throughout, the present invention comprises a system 10 for assessing prospective neuromodulation treatment patients, such as spinal cord stimulation patients, to determine likely patient outcomes and thus inform treatment decisions. System 10 includes a machine learning engine 12 that has been trained using a database 14 of historical patient outcomes along with a set of patient features according to the present invention. A web server 16 facilitates communication with a physician graphical user interface (GUI) 18, a patient GUI 20, and online data storage 22. Referring to FIG. 2, physician GUI 18 allows pain clinicians to enter patient feature data and receive a prediction of likely spinal cord stimulation outcome from machine learning engine 12. Machine learning engine 12 has been trained according to the present invention using historical data in database 14 to perform SCS outcome prediction, as described below, using new patient feature data. As is understood in the art, database 14 may be updated to include outcomes for continuous updating and improvement of machine learning engine 12, such as by identifying new features or changes in the weighting of existing features for more accurate prediction of spinal cord stimulation outcomes.

System 10 is preferably configured as a web-based platform that will be publicly available to physicians who treat and implant patients with SCS. As entering ˜50 data points for an individual patient would be a notable barrier for widespread use, the present invention is configured to focus on about 10-15 of the most important features as optimization and validated as described herein. Different feature selection methods (univariate selection, feature importance, wrapper-based selection, PCA) may be used to select the minimal number of most important features for prediction. Alternatively, imaging segmentation and feature extraction can be substituted for user input. Use of the present invention identified several categories of features and, within each category, specific features that were useful in determining a predicted outcome. Table 1 below highlights the categories and features determined by machine learning engine 12 as useful for predictions.

TABLE 1

| Feature Category | Specific Features |
|---|---|
| Demographics | Age |
| | Sex |
| | Insurance status |
| Pain descriptors | Pain duration |
| | Baseline NRS |
| | Diagnosis |
| | Location |
| Patient questionnaire | McGill pain questionnaire (MPQ) |
| data | Beck Depression inventory (BDI) |
| | Pain catastrophizing scale (PCS) |
| | Oswestry disability index (ODI) |
| Psychiatric | Anxiety |
| comorbidities | Depression |
| | Psychiatric family history |
| Spinal imaging data | Core muscles features (psoas muscle metrics |
| | and sternocleidomastoideole metrics) |
| | Spinal stenosis measurements |
| Patient activity | Work status |
| | ADL |
| Medications | Neuropathic medications |
| | Opioids |
| Non-psychiatric | Charlson index |
| comorbidities | Smoking status |
| | Body mass index (BMI) |
| Historical cpinal | Trial response |
| cord stimulation | |

GUIs may be in Sketch software following clinician' specifications. GUI files will be located and loaded from a server 16, such as Amazon web services (AWS). The GUI for system 10 may be separated into physician 18 and patient GUI 20 so that one GUI can be used by physicians to enter data and one GUI may be used to ask patients to enter data, or provided in a single GUI. As is known in the art, GUIs can require user authentication and login (such as Amazon Cognito) and include input pages, results pages, as well as review and contact pages. An input page may prompt for input of the important features. Machine learning engine 12 will assess the input data and provide, in a preferred embodiment, a prediction of the numeric rating scale (NRS) reduction and global impression of change (GIC) score for a given patient by evaluating the input data against the trained machine learning models developed according to the present invention.

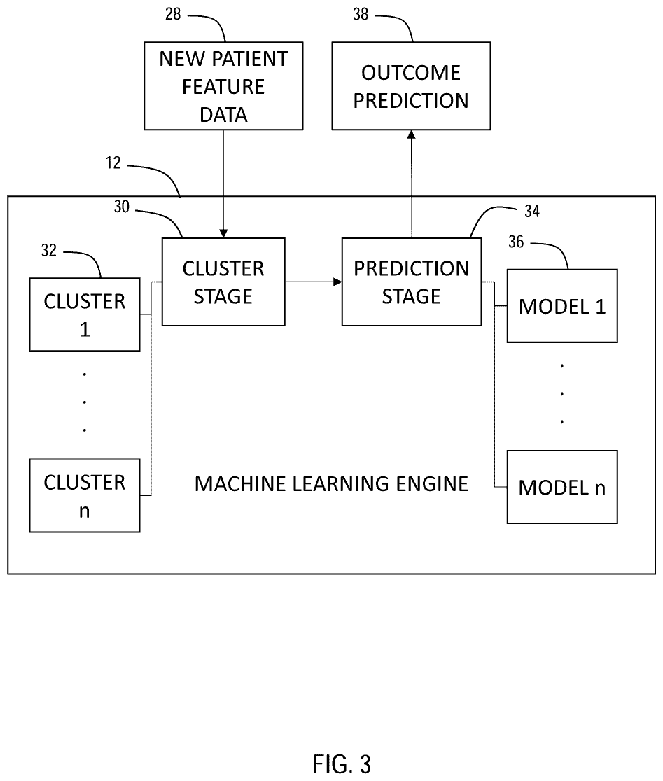

Referring to FIG. 3, a preferred embodiment of machine learning engine 12 of system 10 comprises a cluster identification stage 30 having plurality of clusters 32 that machine learning engine 12 has identified from historical patient features and outcomes in database 14 and that machine learning engine 12 has been trained to use in classifying new patient data 28. Machine learning engine 12 further comprises a prediction stage 34 (called classification) having a plurality of predictive models 36, each of which corresponds to an identified cluster 32 from cluster stage 30. New patient feature data 34 is thus assessed by machine learning engine 12 to determine which predetermined cluster 32 trained into a cluster stage 30 matches the new patient data 34. Once the appropriate cluster 32 is identified by cluster stage 30, prediction stage 34 applies the appropriate predictive model 36 corresponding to the identified cluster of cluster stage 30 to output an outcome prediction 36 for the patient, such as the predicted numeric rating scale (NRS) reduction and global impression of change (GIC) score.

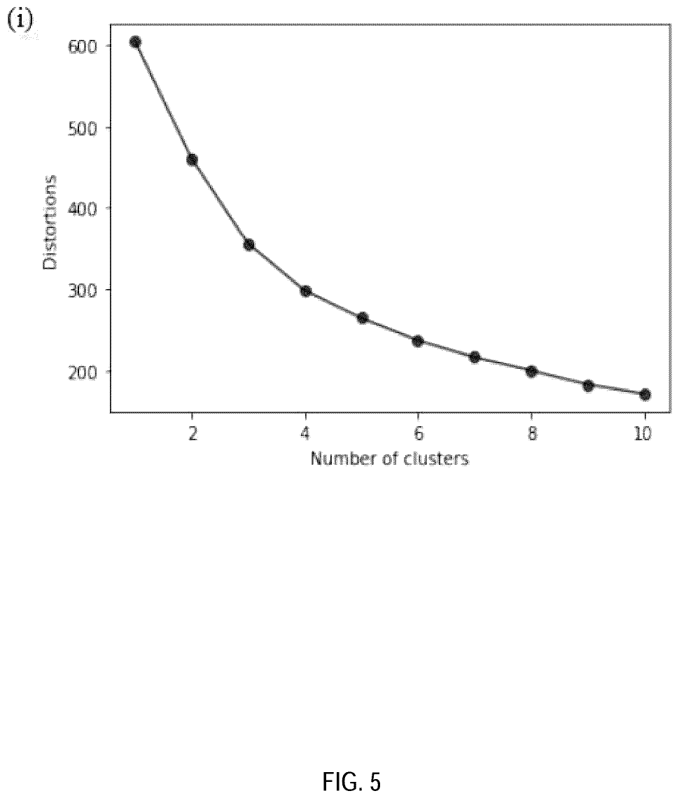

Referring to FIG. 5, the two-phase cluster and model approach of machine learning engine 12 was developed using a combined unsupervised and supervised machine learning approach over two stages. First, the presence of coherent patient clusters/phenotypes was identified. The K-means algorithm was used to discover patient subgroups from a mere data-driven perspective. The K-means algorithm is one of the simplest and most frequently used clustering algorithms. The K-Means clustering uses a simple iterative technique to group points in a dataset into clusters that contain similar characteristics. Initially, a specific number of clusters (K) are decided. The algorithm iteratively places data points into clusters by minimizing the within-cluster sum of squares. The algorithm converges on a solution when either the cluster assignments remain constant or the specified number of iterations is completed.

The patients were clustered based on the following numeric features: age, pain duration (in months), baseline NRS score and baseline PCS total score, based on previous literature pre-dating widespread use of ML. Only numeric features were considered since the K-means algorithm is applicable to numeric features only. Use of the K-modes algorithm, which uses the modes of the clusters instead of means and enables incorporation of additional categorical features, did not improve clustering or classification results and was not used further (data not presented). K-means parameters included random centroid initialization, Euclidian distance similarity metric, and 300 iterations. The elbow method was used to determine the number of clusters (K). Specifically, distortion, defined as the average of the squared distances from the cluster centers, was plotted as function of K. The elbow point in the graph was determined as the maximal number of clusters, as seen in FIG. 5. Although a pseudo elbow was detected at K=3 using Principal Component Analysis (PCA), the computed clusters were projected into two dimensions, and the data space was better separated into two big clusters rather than three.

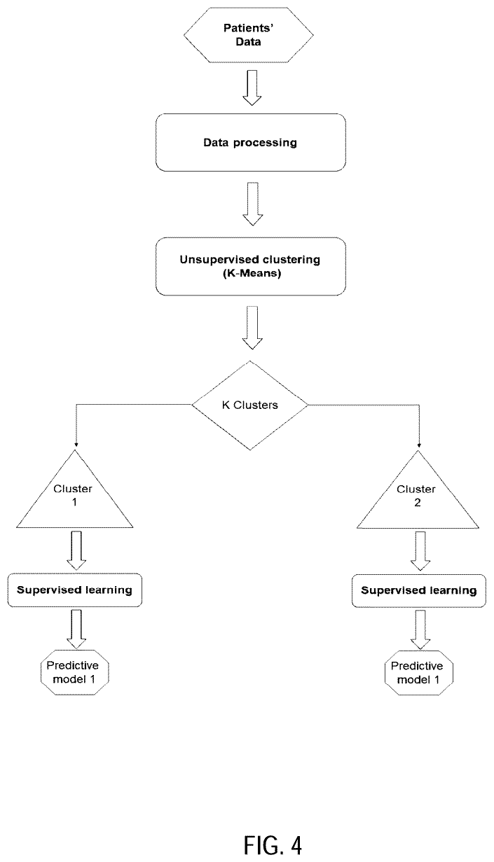

As seen in FIG. 4, the second stage focused on development of ML models for each cluster. Models were developed and prediction performance was evaluated using a nested cross validation (CV) scheme. This approach reduces the overfitting of data and the optimistic bias in error estimation in small sample sizes. Nested CV divides the data set into training and validation components through two separate loops. In the outer loop, the dataset was randomly divided into K=10 folds, meaning that on each iteration, 90% of the data was used to train the model, and 10% of the data was set aside for validation. This was repeated for 10 unique iterations. Missing values imputation using the mean/mode method and numeric features normalization (z=(x-mean)/standard deviation) were performed on each iteration of the outer loop based on 90% of the training data. Due to the significant imbalance of non-high responders to high responders, the synthetic minority oversampling technique (SMOTE) was additionally applied to each loop iteration in the high responder models. In the inner loop, which resides within the training set of the outer loop, the dataset was split into n=10 folds for hyperparameter tuning and feature selection when applied. The term hyperparameter refers to model specific adjustable features that are fine-tuned to obtain a model with optimal performance. Thus, at each iteration of the outer CV, inner CV was repeated for all considered values of hyperparameters, and features were selected accordingly (when applied).

Feature selection was performed using the ten most influential features based on importance weights per model. The values leading to the best inner loop prediction performance were chosen as optimal for that outer loop iteration. Prediction performance was averaged across all outer loop folds. Models tested included logistic regression (LR), Random Forest (RF) and XGBoost. RF are a combination of tree predictors such that each tree depends on the values of a random vector sampled independently and with the same distribution for all trees in the forest. During the training phase, all trees were trained independently. While testing, predictions were made through weighted voting on the most confident predicted class. The trees that had a higher prediction confidence had a greater weight in the final decision of the ensemble. In general, RF shows better predictive performance compared to LR on most datasets. Extreme gradient boosting (XGBoost) is a new gradient boosting ensemble learning method. It implements a ML algorithm under the framework of gradient boosting that can turn weak learners into strong learners and has shown high performance on many standard classification benchmarks. Hyperparameter tuning details are shown in Table 7. For evaluation of the clustering combination, models were developed on the entire cohort. Predictive performance was assessed by the area (AUC) under the receiver operating characteristic curve (ROC), specificity, sensitivity, positive predictive value, and negative predictive value. Overall, clusters models predictive performance were grouped using combined means and standard deviation equations.

Continuous data were expressed as mean±standard deviation. The normal distribution for all variables was tested using the Kolmogorov-Smirnov test. Categorical data were expressed in numbers and percentages. Univariate analysis between responders and non-responders was performed using unpaired t-tests and Chi-Square/Fisher's exact test to identify significant variables. Statistical significance threshold was set to 0.05. Data were statistically analyzed, and models were developed and tested using Python (Python Software Foundation)

### EXAMPLE 1

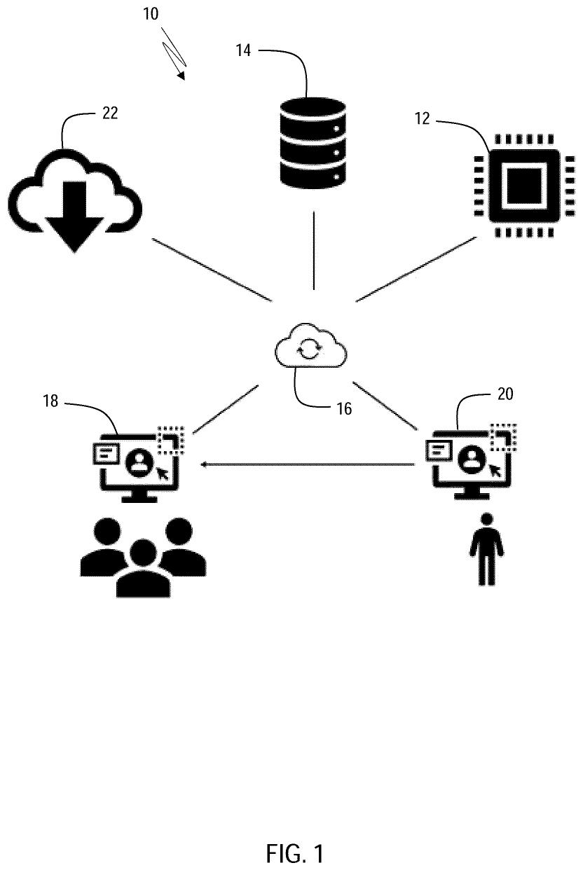

A total of 151 SCS participants with a mean age of 54.8±12.0 were used to evaluate and develop the present invention, as see in FIG. 1. Seventy-eight (51.7%) were treated for failed back surgery syndrome (FBSS), 24 (15.9%) were treated for complex regional pain syndrome (CRPS), and 18 (11.9%) were treated for neuropathic pain. The majority of participants suffered from back (60.3%) and leg pain (70.2%), while 34.4% suffered from pelvic pain and 22.5% had arm pain. While most features had all values, Oswestry disability index (ODI), Beck's depression inventory (BDI) and pain catastrophizing scale (PCS) total scores required imputation of 6%, 13.9% and 11.2% of values, respectively.

Sixty-two participants demonstrated at least a 50% numeric rating scale (NRS) reduction at 1 year (responders), and of those, 31 demonstrated at least a 70% NRS reduction at 1 year (high-responders). The average age was 53.3±11.7 in non-responders compared to 57.0±12.3 in responders (p=0.065). The statistical analysis demonstrated that non-responders more frequently reported arm pain (p=0.003), smoked (p=0.02), and had non-commercial insurance including worker's compensation (p=0.027). Non-responders also had a statistically higher baseline ODI (p=0.014) and Mcgill pain questionnaire (MPQ) score (p=0.029). High responders had a lower body mass index (BMI) (p=0.008) and were less likely to have pelvic (p=0.028), back (p=0.033), or arm pain (p=0.031) than non-responders. They also had lower pre-operative ODI (p=0.004), BDI (p=0.034), MPQ total (p<0.001), and MPQ affective sub score (p=0.014) compared to non-high responders. Additional patient characteristics can be found in Table 2 and Table 3 below.

TABLE 2
Patients' characteristics divided by non-responders and responders

| | | | | | Non- | | | | P- |
|---|---|---|---|---|---|---|---|---|---|
| | | Missing | Total | | Responders | | Responders | | Value |
| N | | | 151 | | 89 | | 62 | | |
| Age, mean (SD) | | 0 | 54.8 | (12.0) | 53.3 | (11.7) | 57.0 | (12.3) | 0.065 |
| Gender, n (%) | Males | 0 | 67 | (44.4) | 36 | (40.4) | 31 | (50.0) | 0.319 |
| | Females | | 84 | (55.6) | 53 | (59.6) | 31 | (50.0) | |
| BMI, mean (SD) | | 0 | 32.2 | (7.6) | 32.7 | (7.7) | 31.4 | (7.4) | 0.327 |
| Pain Duration, | | 3 | 47.8 | (17.5) | 48.3 | (16.8) | 47.2 | (18.5) | 0.718 |
| mean (SD) | | | | | | | | | |
| Pelvis pain, n (%) | | | 52 | (34.4) | 36 | (40.4) | 16 | (25.8) | 0.091 |
| Back pain, n (%) | | | 91 | (60.3) | 58 | (65.2) | 33 | (53.2) | 0.191 |
| Neck pain, n (%) | | | 28 | (18.5) | 20 | (22.5) | 8 | (12.9) | 0.202 |
| Legs pain, n (%) | | | 106 | (70.2) | 66 | (74.2) | 40 | (64.5) | 0.274 |
| Arms pain, n (%) | | | 34 | (22.5) | 28 | (31.5) | 6 | (9.7) | 0.003 |
| Diagnosis, n (%) | FBSS | 0 | 78 | (51.7) | 44 | (49.4) | 34 | (54.8) | 0.732 |
| | CRPS | | 24 | (15.9) | 14 | (15.7) | 10 | (16.1) | |
| | Neuropathy | | 18 | (11.9) | 10 | (11.2) | 8 | (12.9) | |
| | Other | | 31 | (20.5) | 21 | (23.6) | 10 | (16.1) | |
| Psychiatric family | None | 0 | 142 | (94.0) | 81 | (91.0) | 61 | (98.4) | 0.082 |
| history, n (%) | Yes | | 9 | (6.0) | 8 | (9.0) | 1 | (1.6) | |
| Anxiety, n (%) | None | 0 | 115 | (76.2) | 67 | (75.3) | 48 | (77.4) | 0.956 |
| | Mild | | 32 | (21.2) | 20 | (22.5) | 12 | (19.4) | |
| | Moderate | | 2 | (1.3) | 1 | (1.1) | 1 | (1.6) | |
| | Severe | | 2 | (1.3) | 1 | (1.1) | 1 | (1.6) | |
| Depression, n (%) | None | 0 | 97 | (64.2) | 54 | (60.7) | 43 | (69.4) | 0.434 |
| | Mild | | 49 | (32.5) | 33 | (37.1) | 16 | (25.8) | |
| | Moderate | | 3 | (2.0) | 1 | (1.1) | 2 | (3.2) | |
| | Severe | | 2 | (1.3) | 1 | (1.1) | 1 | (1.6) | |
| Smoking, n (%) | Current | 0 | 67 | (44.4) | 46 | (51.7) | 21 | (33.9) | 0.020 |
| | Never | | 43 | (28.5) | 18 | (20.2) | 25 | (40.3) | |
| | Former | | 41 | (27.2) | 25 | (28.1) | 16 | (25.8) | |
| Insurance, n (%) | Commercial | 0 | 110 | (72.8) | 57 | (64.0) | 53 | (85.5) | 0.027 |
| | Medicare | | 3 | (2.0) | 3 | (3.4) | | | |
| | No Fault | | 5 | (3.3) | 4 | (4.5) | 1 | (1.6) | |
| | (Auto) | | | | | | | | |
| | Workers | | 33 | (21.9) | 25 | (28.1) | 8 | (12.9) | |
| | Comp | | | | | | | | |
| Previous spinal | | 0 | 1.3 | (1.6) | 1.2 | (1.2) | 1.3 | (1.9) | 0.815 |
| surgeries (SD) | | | | | | | | | |
| Months from | | 0 | 80.6 | (101.0) | 78.4 | (97.3) | 85.1 | (109.8) | 0.79 |
| previous surgery* | | | | | | | | | |
| (SD) | | | | | | | | | |
| NRS Baseline, | | 0 | 6.9 | (1.7) | 7.1 | (1.9) | 6.7 | (1.5) | 0.084 |
| mean (SD) | | | | | | | | | |
| ODI_Baseline, | | 10 | 25.2 | (7.2) | 26.4 | (6.8) | 23.3 | (7.5) | 0.014 |
| mean (SD) | | | | | | | | | |
| BDI_Baseline, | | 21 | 13.3 | (9.0) | 14.5 | (9.6) | 11.6 | (7.8) | 0.056 |
| mean (SD) | | | | | | | | | |
| PCSTotal_Baseline, | | 17 | 23.2 | (12.9) | 24.2 | (13.0) | 21.7 | (12.6) | 0.269 |
| mean (SD) | | | | | | | | | |
| MPQTotal_Baseline, | | 0 | 5.2 | (2.8) | 5.6 | (2.7) | 4.6 | (2.9) | 0.029 |
| mean (SD) | | | | | | | | | |
| MPQAffective_Baseline, | | 0 | 0.7 | (0.9) | 0.8 | (0.9) | 0.6 | (1.0) | 0.330 |
| mean (SD) | | | | | | | | | |

N = Sample size. BMI = Body mass index. FBSS = failed back surgery syndrome, CRPS = complex regional pain syndrome, NRS = numeric rating scale, ODI = Oswestry disability index, BDI = Beck's depression inventory, PCS = pain catastrophizing scale, MPQ = McGill pain questionnaire, *only including patients with at least one previous surgery. The statistically significant differences are highlighted in gray and bold.

TABLE 3
Patients' characteristics divided by high responders vs non-high responders.

| | | | | | Non-High | | High | | P- |
|---|---|---|---|---|---|---|---|---|---|
| | | Missing | Total | | Responders | | responders | | Value |
| N | | | 151 | | 120 | | 31 | | |
| Age, mean (SD) | | 0 | 54.8 | (12.0) | 53.9 | (11.5) | 58.2 | (13.5) | 0.111 |
| Gender, n (%) | Males | 0 | 67 | (44.4) | 57 | (47.5) | 10 | (32.3) | 0.187 |
| | Females | | 84 | (55.6) | 63 | (52.5) | 21 | (67.7) | |
| BMI, mean (SD) | | 0 | 32.2 | (7.6) | 32.9 | (7.8) | 29.2 | (6.3) | 0.008 |
| Pain Duration, | | 3 | 47.8 | (17.5) | 47.8 | (17.2) | 47.8 | (18.8) | 0.999 |
| mean (SD) | | | | | | | | | |
| Pelvis pain, n(%) | | | 52 | (34.4) | 47 | (39.2) | 5 | (16.1) | 0.028 |
| Back pain, n(%) | | | 91 | (60.3) | 78 | (65.0) | 13 | (41.9) | 0.033 |
| Neck pain, n(%) | | | 28 | (18.5) | 25 | (20.8) | 3 | (9.7) | 0.244 |
| Legs pain, n(%) | | | 106 | (70.2) | 89 | (74.2) | 17 | (54.8) | 0.06 |
| Arms pain, n(%) | | | 34 | (22.5) | 32 | (26.7) | 2 | (6.5) | 0.031 |
| Diagnosis, n (%) | FBSS | 0 | 78 | (51.7) | 62 | (51.7) | 16 | (51.6) | 0.169 |
| | CRPS | | 24 | (15.9) | 20 | (16.7) | 4 | (12.9) | |
| | Neuropathy | | 18 | (11.9) | 11 | (9.2) | 7 | (22.6) | |
| | Other | | 31 | (20.5) | 27 | (22.5) | 4 | (12.9) | |
| Psychiatric family | None | 0 | 142 | (94.0) | 111 | (92.5) | 31 | (100.0) | 0.205 |
| history, n (%) | Yes | | 9 | (6.0) | 9 | (7.5) | | | |
| Anxiety, n (%) | None | 0 | 115 | (76.2) | 89 | (74.2) | 26 | (83.9) | 0.613 |
| | Mild | | 32 | (21.2) | 27 | (22.5) | 5 | (16.1) | |
| | Moderate | | 2 | (1.3) | 2 | (1.7) | | | |
| | Severe | | 2 | (1.3) | 2 | (1.7) | | | |
| Depression, n (%) | None | 0 | 97 | (64.2) | 77 | (64.2) | 20 | (64.5) | 0.138 |
| | Mild | | 49 | (32.5) | 41 | (34.2) | 8 | (25.8) | |
| | Moderate | | 3 | (2.0) | 1 | (0.8) | 2 | (6.5) | |
| | Severe | | 2 | (1.3) | 1 | (0.8) | 1 | (3.2) | |
| Smoking, n (%) | Current | 0 | 67 | (44.4) | 56 | (46.7) | 11 | (35.5) | 0.068 |
| | Never | | 43 | (28.5) | 29 | (24.2) | 14 | (45.2) | |
| | Former | | 41 | (27.2) | 35 | (29.2) | 6 | (19.4) | |
| Insurance, n (%) | Commercial | 0 | 110 | (72.8) | 85 | (70.8) | 25 | (80.6) | 0.640 |
| | Medicare | | 3 | (2.0) | 3 | (2.5) | | | |
| | No Fault | | 5 | (3.3) | 4 | (3.3) | 1 | (3.2) | |
| | (Auto) | | | | | | | | |
| | Workers | | 33 | (21.9) | 28 | (23.3) | 5 | (16.1) | |
| | Comp | | | | | | | | |
| Previous spinal | | 0 | 1.3 | (1.6) | 1.3 | (1.5) | 1.2 | (1.7) | 0.923 |
| surgeries (SD) | | | | | | | | | |
| Months from previous | | 0 | 80.6 | (101.0) | 81.1 | (103.8) | 78.3 | (88.2) | 0.921 |
| surgery (SD)* | | | | | | | | | |
| AverageNRS_Baseline, | | 0 | 6.9 | (1.7) | 7.0 | (1.8) | 6.8 | (1.6) | 0.529 |
| mean (SD) | | | | | | | | | |
| ODI_Baseline, | | 10 | 25.2 | (7.2) | 26.1 | (7.1) | 21.7 | (6.9) | 0.004 |
| mean (SD) | | | | | | | | | |
| BDI_Baseline, | | 21 | 13.3 | (9.0) | 14.1 | (9.2) | 10.3 | (7.3) | 0.034 |
| mean (SD) | | | | | | | | | |
| PCSTotal_Baseline, | | 17 | 23.2 | (12.9) | 23.6 | (13.0) | 21.5 | (12.4) | 0.448 |
| mean (SD) | | | | | | | | | |
| MPQTotal_Baseline, | | 0 | 5.2 | (2.8) | 5.6 | (2.7) | 3.6 | (2.4) | <0.001 |
| mean (SD) | | | | | | | | | |
| MPQAffective_Baseline, | | 0 | 0.7 | (0.9) | 0.8 | (1.0) | 0.4 | (0.7) | 0.014 |
| mean (SD) | | | | | | | | | |

N = Sample size. BMI = Body mass index. FBSS = failed back surgery syndrome, CRPS = complex regional pain syndrome, NRS = numeric rating scale, ODI = Oswestry disability index, BDI = Beck's depression inventory, PCS = pain catastrophizing scale, MPQ = McGill pain questionnaire, *only including patients with at least one previous surgery. The statistically significant differences are highlighted in gray and bold.

### Clustering

Following K-means clustering optimization, two distinct clusters (Cluster 1: n=79, Cluster 2: n=72) were found (Table 4 below). As expected, there were significant differences between the clusters. Cluster 1 included patients who were younger (51.5±11.8 vs.58.5±11.2, p<0.001), had shorter pain duration (43.8±17.5 vs 52.3±13.8, p=0.002), had higher baseline NRS (7.9±1.3 vs. 5.8±1.4, p<0.001) and higher PCS total scores (32.0±9.7 vs. 14.1±18.7, p<0.001) compared to cluster 2. In addition, patients in cluster 1 had a lower number of previous spinal surgeries (0.9±1.2 vs. 1.6±1.8, p=0.005), higher BDI scores (17.0±9.6 vs. 9.6±6.6, p<0.001), higher ODI scores (27.5±6.3 vs. 22.6±7.4, p<0.001) and higher rates of CRPS (24.1% vs 6.9%, p=0.008). Notably, both clusters had similar rates of responders (36.7% in cluster 1 and 45.8% in cluster 2) and high-responders (17.7% in cluster 1 and 23.6% in cluster 2) (Table 4 below).

TABLE 4
Patients' characteristics divided by the two distinct clusters

| | | Missing | Total | | Cluster 1 | | Cluster 2 | | P-Value |
|---|---|---|---|---|---|---|---|---|---|
| M | | | 151 | | 79 | | 72 | | |
| Responder, n (%) | | | 62 | | 29 | (36.7%) | 33 | (45.8%) | 0.331 |
| High responder, n (%) | | | 31 | (20.5) | 14 | (17.7) | 17 | (23.6) | 0.488 |
| Age, mean (SD) | | 0 | 54.8 | (12.0) | 51.5 | (11.8) | 58.5 | (11.2) | <0.001 |
| Gender (%) | Females | 0 | 84 | (55.6) | 48 | (60.8) | 36 | (50.0) | 0.244 |
| | Males | | 67 | (44.4) | 31 | (39.2) | 36 | (50.0) | |
| BMI, mean (SD) | | 0 | 32.2 | (7.6) | 32.3 | (7.7) | 32.0 | (7.6) | 0.838 |
| PainDuration, mean (SD) | | 3 | 47.8 | (17.5) | 43.8 | (19.4) | 52.3 | (13.8) | 0.002 |
| Pelvis_Baseline, n (%) | | | 52 | (34.4) | 29 | (36.7) | 23 | (31.9) | 0.657 |
| Back_Baseline, n (%) | | | 91 | (60.3) | 46 | (58.2) | 45 | (62.5) | 0.712 |
| Neck_Baseline, n (%) | | | 28 | (18.5) | 12 | (15.2) | 16 | (22.2) | 0.368 |
| Legs_Baseline, n (%) | | | 106 | (70.2) | 57 | (72.2) | 49 | (68.1) | 0.710 |
| Arms_Baseline, n (%) | | | 34 | (22.5) | 18 | (22.8) | 16 | (22.2) | 0.911 |
| Diagnosis (%) | FBSS | | 78 | (51.7) | 34 | (43.0) | 44 | (61.1) | 0.04 |
| | CRPS | | 24 | (15.9) | 19 | (24.1) | 5 | (6.9) | 0.008 |
| | Neuropathy | | 18 | (11.9) | 8 | (10.1) | 10 | (13.9) | 0.645 |
| | Other | | 31 | (20.5) | 18 | (22.8) | 13 | (18.1) | 0.605 |
| Psychiatric family | None | 0 | 142 | (94.0) | 74 | (93.7) | 68 | (94.4) | 1.000 |
| history, n (%) | Yes | | 9 | (6.0) | 5 | (6.3) | 4 | (5.6) | |
| Anxiety, n (%) | None | 0 | 115 | (76.2) | 58 | (73.4) | 57 | (79.2) | 0.534 |
| | Mild | | 32 | (21.2) | 18 | (22.8) | 14 | (19.4) | |
| | Moderate | | 2 | (1.3) | 2 | (2.5) | | | |
| | Severe | | 2 | (1.3) | 1 | (1.3) | 1 | (1.4) | |
| Depression, n (%) | None | 0 | 97 | (64.2) | 48 | (60.8) | 49 | (68.1) | 0.361 |
| | Mild | | 49 | (32.5) | 27 | (34.2) | 22 | (30.6) | |
| | Moderate | | 3 | (2.0) | 3 | (3.8) | | | |
| | Severe | | 2 | (1.3) | 1 | (1.3) | 1 | (1.4) | |
| Smoking (%) | Never Smoking | | 67 | (44.4) | 38 | (48.1) | 29 | (40.3) | |
| | Former | | 43 | (28.5) | 16 | (20.3) | 27 | (37.5) | 0.030 |
| | Smoking | | | | | | | | |
| | Current | | 41 | (27.2) | 25 | (31.6) | 16 | (22.2) | |
| | Smoking | | | | | | | | |
| Insurance (%) | Medicare | | 3 | (2.0) | | | 3 | (4.2) | 0.106 |
| | Commercial | | 110 | (72.8) | 56 | (70.9) | 54 | (75.0) | 0.701 |
| | NoFault | | 5 | (3.3) | 3 | (3.8) | 2 | (2.8) | 1 |
| | Worker | | 33 | (21.9) | 20 | (25.3) | 13 | (18.1) | 0.378 |
| | Compensation | | | | | | | | |
| Previous spinal | | 0 | 1.3 | (1.6) | 0.9 | (1.2) | 1.6 | (1.8) | 0.005 |
| surgeries (SD) | | | | | | | | | |
| Months from previous | | 0 | 80.6 | (101) | 66.1 | (75.8) | 92.9 | (117.5) | 0.22 |
| surgery* (SD) | | | | | | | | | |
| NRS Baseline, mean (SD) | | 0 | 6.9 | (1.7) | 7.9 | (1.3) | 5.8 | (1.4) | <0.001 |
| ODI_Baseline, mean (SD) | | 10 | 25.2 | (7.2) | 27.5 | (6.3) | 22.6 | (7.4) | <0.001 |
| BDI_Baseline, mean (SD) | | 21 | 13.3 | (9.0) | 17.0 | (9.6) | 9.6 | (6.6) | <0.001 |
| PCSTotal_Baseline, | | 17 | 23.2 | (12.9) | 32.0 | (9.7) | 14.1 | (8.7) | <0.001 |
| mean (SD) | | | | | | | | | |
| MPQTotal_Baseline, | | 0 | 5.2 | (2.8) | 5.6 | (2.9) | 4.8 | (2.6) | 0.048 |
| mean (SD) | | | | | | | | | |
| MPQAffective_Baseline, | | 0 | 0.7 | (0.9) | 0.8 | (1.0) | 0.6 | (0.9) | 0.167 |
| mean (SD) | | | | | | | | | |

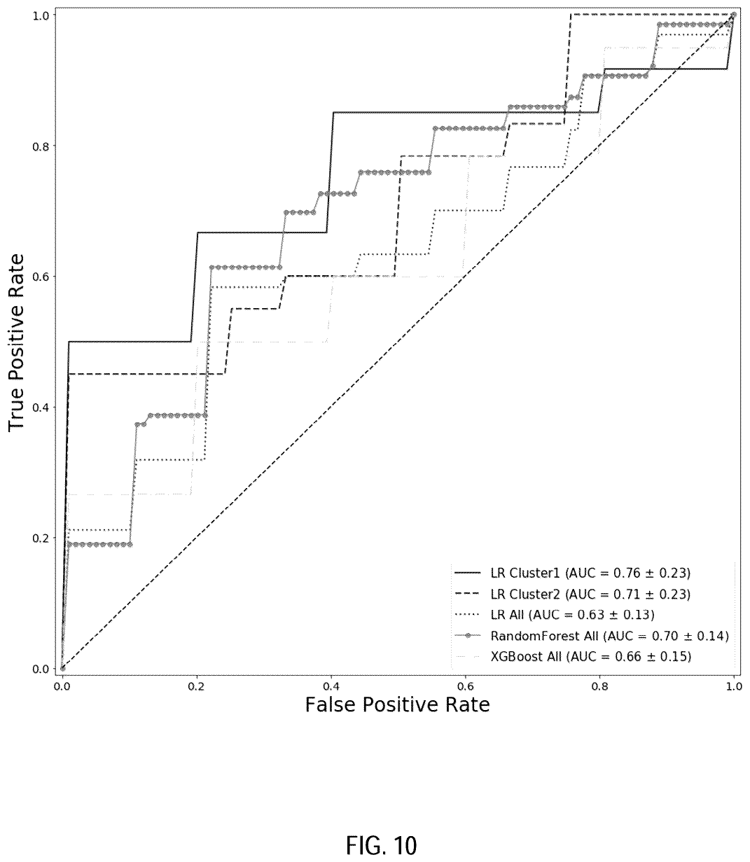

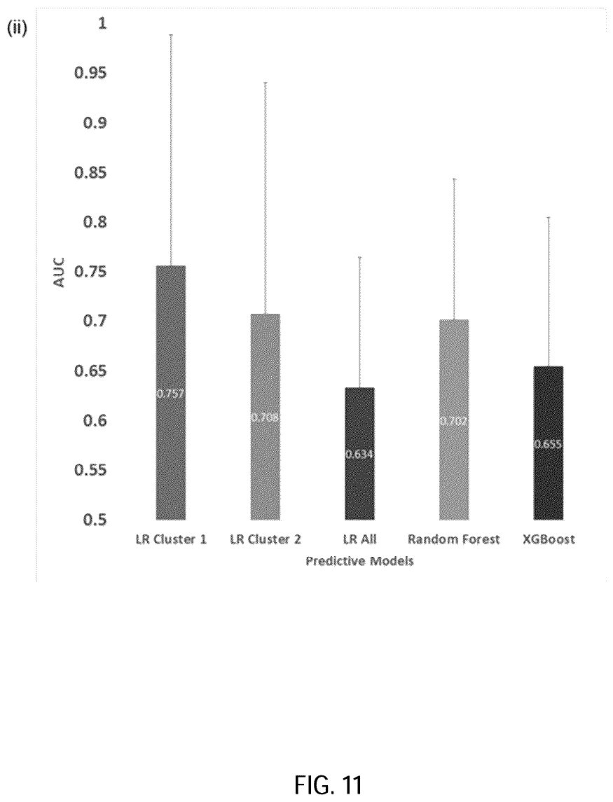

Internally validated performances of the ML predictive models for responders are summarized in Table 4 and FIGS. 10 and 11. When all 31 features were used to predict the responders in cluster 1, best performance was obtained with LR model with an AUC of 0.757, sensitivity of 61.7%, specificity of 80%, and accuracy of 73.4%. When the features were downsized to the 10 most important features (see Table 8), overall performance remained high with AUC of 0.757 while sensitivity decreased to 50%. Responders in cluster 2 were best predicted by LR model using the 10 most important features (see Table 8) with AUC of 0.708, sensitivity of 63.3%, specificity 61.7% and accuracy of 62%. The combination of the separate performances of the LR models based on the 10 most important features in the two clusters showed higher performance than that of the model based on the entire cohort (AUC: 0.732 vs 0.653, respectively). The performance of both RF and XGBoost models on the entire cohort were higher than that performed on individual clusters or the combination of the clusters' separate performances (AUC: 0.706 and 0.655 respectively) (see Table 5 below).

TABLE 5
Performance comparison of predictive models: responders

| Algorithms | Clusters | AUC | Sensitivity | Specificity | PPV | NPV | Accuracy |
|---|---|---|---|---|---|---|---|
| Logistic | | | | | | | |
| Regression | | | | | | | |
| All | 1 | 0.757 | 0.617 | 0.800 | 0.658 | 0.790 | 0.734 |
| features | | (0.213) | (0.193) | (0.133) | (0.202) | (0.095) | (0.110) |
| | 2 | 0.608 | 0.592 | 0.642 | 0.517 | 0.725 | 0.621 |
| | | (0.297) | (0.382) | (0.171) | (0.285) | (0.224) | (0.171) |
| | Combination | 0.682 | 0.604 | 0.721 | 0.587 | 0.757 | 0.677 |
| | | (0.262) | (0.294) | (0.169) | (0.251) | (0.170) | (0.151) |
| | Whole | 0.638 | 0.400 | 0.760 | 0.570 | 0.642 | 0.615 |
| | cohort | (0.144) | (0.154) | (0.177) | (0.209) | (0.110) | (0.131) |
| Logistic | | | | | | | |
| Regression | | | | | | | |
| 10 features | 1 | 0.757 | 0.500 | 0.900 | 0.658 | 0.783 | 0.759 |
| | | (0.232) | (0.360) | (0.141) | (0.390) | (0.125) | (0.110) |
| | 2 | 0.708 | 0.633 | 0.617 | 0.550 | 0.710 | 0.620 |
| | | (0.233) | (0.343) | (0.172) | (0.270) | (0.236) | (0.207) |
| | Combination | 0.732 | 0.566 | 0.758 | 0.604 | 0.746 | 0.689 |
| | | (0.227) | (0.349) | (0.211) | (0.331) | (0.187) | (0.176) |
| | Whole | 0.653 | 0.371 | 0.778 | 0.422 | 0.654 | 0.609 |
| | cohort | (0.146) | (0.276) | (0.128) | (0.243) | (0.085) | (0.076) |
| Random | | | | | | | |
| Forest | | | | | | | |
| All | 1 | 0.710 | 0.367 | 0.900 | 0.583 | 0.723 | 0.707 |
| features | | (0.233) | (0.331) | (0.141) | (0.466) | (0.131) | (0.171) |
| | 2 | 0.550 | 0.433 | 0.750 | 0.517 | 0.620 | 0.600 |
| | | (0.220) | (0.288) | (0.204) | (0.309) | (0.167) | (0.197) |
| | Combination | 0.630 | 0.400 | 0.825 | 0.550 | 0.671 | 0.683 |
| | | (0.235) | (0.303) | (0.187) | (0.386) | (0.155) | (0.181) |
| | Whole | 0.706 | 0.431 | 0.794 | 0.571 | 0.677 | 0.648 |
| | cohort | (0.192) | (0.267) | (0.139) | (0.230) | (0.126) | (0.142) |
| Random | | | | | | | |
| Forest | | | | | | | |
| 10 features | 1 | 0.550 | 0.233 | 0.760 | 0.370 | 0.607 | 0.570 |
| | | (0.291) | (0.274) | (0.310) | (0.462) | (0.190) | (0.244) |
| | 2 | 0.500 | 0.183 | 0.750 | 0.333 | 0.515 | 0.487 |
| | | (0.297) | (0.254) | (0.264) | (0.471) | (0.150) | (0.217) |
| | Combination | 0.525 | 0.208 | 0.755 | 0.351 | 0.561 | 0.528 |
| | | (0.287) | (0.258) | (0.280) | (0.454) | (0.173) | (0.228) |
| | Whole | 0.697 | 0.443 | 0.796 | 0.548 | 0.686 | 0.655 |
| | cohort | (0.098) | (0.249) | (0.142) | (0.214) | (0.062) | (0.062) |
| XGBoost | | | | | | | |
| All | 1 | 0.657 | 0.550 | 0.760 | 0.607 | 0.746 | 0.684 |
| features | | (0.185) | (0.273) | (0.207) | (0.330) | (0.137) | (0.118) |
| | 2 | 0.477 | 0.433 | 0.550 | 0.492 | 0.540 | 0.407 |
| | | (0.272) | (0.235) | (0.307) | (0.287) | (0.220) | (0.195) |
| | Combination | 0.567 | 0.491 | 0.655 | 0.549 | 0.643 | 0.545 |
| | | (0.244) | (0.255) | (0.276) | (0.306) | (0.207) | (0.211) |
| | Whole | 0.655 | 0.476 | 0.662 | 0.488 | 0.650 | 0.583 |
| | cohort | (0.150) | (0.223) | (0.139) | (0.152) | (0.138) | (0.136) |
| XGBoost | | | | | | | |
| 10 features | 1 | 0.607 | 0.433 | 0.760 | 0.525 | 0.704 | 0.643 |
| | | (0.343) | (0.316) | (0.207) | (0.389) | (0.148) | (0.201) |
| | 2 | 0.423 | 0.392 | 0.550 | 0.457 | 0.497 | 0.475 |
| | | (0.230) | (0.125) | (0.258) | (0.216) | (0.134) | (0.145) |
| | Combination | 0.515 | 0.412 | 0.655 | 0.491 | 0.600 | 0.559 |
| | | (0.299) | (0.234) | (0.251) | (0.308) | (0.173) | (0.191) |
| | Whole | 0.652 | 0.486 | 0.708 | 0.530 | 0.675 | 0.616 |
| | cohort | (0.065) | (0.206) | (0.106) | (0.049) | (0.086 | (0.058) |

AUC = area under the curve,
PPV = positive predictive value,
NPV = negative predictive value

Internally validated performances of the ML predictive models for high responders are summarized in Table 6 below. Similarly, best model performance to predict high responders in each cluster were obtained with LR model using the 10 most important features (AUC 0.729 in cluster 1 and AUC 0.647 in cluster 2). LR using their combined performance with 10 features showed AUC of 0.688. sensitivity of 57.5%. specificity 79.6% and 74.2% accuracy. These were higher than LR model when used on the entire cohort.

TABLE 6
Performance comparison of predictive models: high-responders

| Algorithms | Clusters | AUC | Sensitivity | Specificity | PPV | NPV | Accuracy |
|---|---|---|---|---|---|---|---|
| Logistic | | | | | | | |
| Regression | | | | | | | |
| All features | 1 | 0.737 | 0.450 | 0.826 | 0.350 | 0.867 | 0.761 |
| | | (0.218) | (0.438) | (0.157) | (0.388) | (0.108) | (0.170) |
| | 2 | 0.593 | 0.450 | 0.737 | 0.400 | 0.787 | 0.655 |
| | | (0.320) | (0.438) | (0.247) | (0.394) | (0.178) | (0.252) |
| | Combination | 0.665 | 0.450 | 0.781 | 0.375 | 0.827 | 0.708 |
| | | (0.276) | (0.426) | (0.206) | (0.381) | (0.149) | (0.468) |
| | Whole | 0.683 | 0.467 | 0.783 | 0.363 | 0.854 | 0.716 |
| | cohort | (0.212) | (0.358) | (0.181) | (0.303) | (0.095) | (0.154 |
| Logistic | | | | | | | |
| Regression | | | | | | | |
| 10 features | 1 | 0.729 | 0.550 | 0.805 | 0.358 | 0.907 | 0.761 |
| | | (0.212) | (0.438) | (0.139) | (0.314) | (0.081) | (0.090) |
| | 2 | 0.647 | 0.600 | 0.787 | 0.442 | 0.857 | 0.723 |
| | | (0.351) | (0.394) | (0.129) | (0.299) | (0.135) | (0.139) |
| | Combination | 0.688 | 0.575 | 0.796 | 0.400 | 0.882 | 0.742 |
| | | (0.285) | (0.406) | (0.130) | (0.301) | (0.111) | (0.115) |
| | Whole | 0.653 | 0.467 | 0.733 | 0.336 | 0.845 | 0.676 |
| | cohort | (0.155) | (0.322) | (0.146) | (0.293) | (0.089) | (0.124) |
| Random | | | | | | | |
| Forest | | | | | | | |
| All features | 1 | 0.727 | 0.300 | 0.907 | 0.300 | 0.858 | 0.796 |
| | | (0.212) | (0.422) | (0.081) | (0.422) | (0.091) | (0.108) |
| | 2 | 0.537 | 0.200 | 0.843 | 0.133 | 0.780 | 0.682 |
| | | (0.336) | (0.350) | (0.125) | (0.219) | (0.091) | (0.054) |
| | Combination | 0.632 | 0.250 | 0.875 | 0.216 | 0.819 | 0.739 |
| | | (0.290) | (0.380) | (0.107) | (0.338) | (0.097) | (0.101) |
| | Whole | 0.622 | 0.300 | 0.892 | 0.350 | 0.832 | 0.768 |
| | cohort | (0.203) | (0.246) | (0.097) | (0.326) | (0.056) | (0.090) |
| Random | | | | | | | |
| Forest | | | | | | | |
| 10 features | 1 | 0.683 | 0.400 | 0.893 | 0.350 | 0.868 | 0.798 |
| | | (0.218) | (0.459) | (0.101) | (0.412) | (0.107) | (0.133) |
| | 2 | 0.547 | 0.250 | 0.747 | 0.158 | 0.773 | 0.623 |
| | | (0.249) | (0.354) | (0.161) | (0.217) | (0.097) | (0.100) |
| | Combination | 0.615 | 0.325 | 0.820 | 0.254 | 0.820 | 0.710 |
| | | (0.238) | (0.406) | (0.151) | (0.335) | (0.110) | (0.145) |
| | Whole | 0.568 | 0.292 | 0.900 | 0.382 | 0.833 | 0.775 |
| | cohort | (0.141) | (0.246) | (0.102) | (0.371) | (0.050) | (0.082) |
| XGBoost | | | | | | | |
| All features | 1 | 0.717 | 0.350 | 0.829 | 0.267 | 0.857 | 0.746 |
| | | (0.251) | (0.412) | (0.119) | (0.335) | (0.088) | (0.118) |
| | 2 | 0.475 | 0.250 | 0.750 | 0.183 | 0.770 | 0.627 |
| | | (0.202) | (0.354) | (0.107) | (0.242) | (0.103) | (0.105) |
| | Combination | 0.596 | 0.300 | 0.789 | 0.225 | 0.813 | 0.686 |
| | | (0.254) | (0.374) | (0.117) | (0.287) | (0.103) | (0.124) |
| | Whole | 0.613 | 0.333 | 0.850 | 0.382 | 0.871 | 0.742 |
| | cohort | (0.234) | (0.314) | (0.135) | (0.379) | (0.079) | (0.134) |
| XGBoost | | | | | | | |
| 10 features | 1 | 0.687 | 0.500 | 0.800 | 0.308 | 0.870 | 0.736 |
| | | (0.324) | (0.471) | (0.143) | (0.329) | (0.125) | (0.159) |
| | 2 | 0.475 | 0.250 | 0.750 | 0.183 | 0.770 | 0.627 |
| | | (0.202) | (0.354) | (0.107) | (0.242) | (0.103) | (0.105 |
| | Combination | 0.581 | 0.375 | 0.775 | 0.245 | 0.820 | 0.681 |
| | | (0.284) | (0.425) | (0.125) | (0.288) | (0.122) | (0.142) |
| | Whole | 0.628 | 0.267 | 0.858 | 0.337 | 0.818 | 0.736 |
| | cohort | (0.175) | (0.211) | (0.131) | (0.373) | (0.058) | (0.117) |

AUC = area under the curve,
PPV = positive predictive value,
NPV = negative predictive value

The present example demonstrated, for the first time, the ability of ML derived algorithms to predict long-term patient response to SCS placement with relatively high performance (0.708-0.757 AUC for prediction of responders; 0.647-0.729 AUC for prediction of high responders). The nested cross validation (CV) method used for internal validation provided a true estimate of the generalized performance of our models. In addition, the study demonstrated how the combination of unsupervised and supervised learning can develop patient individualized models based on predicted clusters to increase overall predictive performance (0.757 and 0.708 for the clusters and 0.706 for the entire cohort).

Although the present invention used sensitivity, specificity, and accuracy statistics (similarly to previous studies), these measures can be problematic since they depend on a diagnostic criterion for positivity which is often chosen arbitrarily. For example, the model predicted a probability for a certain outcome to occur (e.g., high responder); if that probability was higher than a standard threshold of 0.5, that patient was labeled as a higher responder. However, one observer may choose a more lenient decision criterion and the other may choose a more stringent decision criterion for positivity. Thus, sensitivity, specificity and accuracy may vary across the different thresholds. In the current example, 0.5 was used as the standard threshold. The area under the curve (AUC) of a receiver operating curve (ROC) circumvented this arbitrary threshold and provided a more effective method to evaluate predictive performance between different models. Thus, the models of the present invention provided relatively high overall performance of 0.64-0.76. Moreover, the models reported the probabilities for a responder/high-responder, and ultimately would allow the clinician to decide on the threshold.

Using the unsupervised approach as a first stage, two distinct clusters were found based on patients' age, pain duration, baseline NRS, and baseline PCS total scores. All of these scores have been previously associated with SCS outcomes, but have not been clustered using the ML techniques herein. These clusters likely represent two distinct SCS populations: younger patients with higher pain scores who have been suffering for a shorter duration and an older population with longer chronic pain duration with lower pain scores. Although there were no significant differences in response rates between the two clusters, each cluster required an individualized model and different set of selected features to provide optimized performance, suggesting two different phenotypes.

Through hyperparameter fine-tuning and supervised intrinsic feature selection, the ten most influential features that contribute the most to the model performance were identified. Several of these features, including presence of depression, number of previous spinal surgeries, BMI, insurance type, and smoking status, have been documented as predictors of poor response in the literature. For example, psychological factors, including somatization, depression, and anxiety have been established as poor prognostic markers of outcome such that pre-operative psychological testing has become the standard of care for SCS placement. Current smoking status has also been statistically associated with decreased NRS reduction compared to former and non-smokers. Published data have also demonstrated poor outcomes of SCS in a worker's compensation setting, showing comparable results of SCS to conservative pain management therapies. The congruency of these selected features with characteristics identified in prior studies substantiate the validity of our ML derived models. Moreover, identification of these features in our model can help guide preoperative optimization by addressing these modifiable patient factors to increase the chance of clinical success. Ultimately, these factors likely represent confounders that complicate the underlying pathophysiology, and processing of chronic pain through a mechanism research has yet to fully elucidate. While numerous studies have identified various patient characteristics and demographic features associated with improved SCS outcomes in effort to tailor patient selection the present invention was the first to provide reasonably high-performance ML based algorithms.

The combined unsupervised-supervised ML approach yielded relatively high predictive performance for long term SCS outcomes in chronic pain patients. The clustering technique enabled finer individualized predictions for patients who share a common set of features. Each cluster used a unique model with a different set of features for optimal predictions. ML models of SCS response may be integrated to clinical routine and used to augment, not replace, clinical judgement. The present invention thus suggests that the advanced ML derived approaches have the potential to be utilized as a functional clinical tool to improve SCS outcomes.

The study protocol was approved by Albany Medical Center Institutional Review Board. Data were collected prospectively and longitudinally except where otherwise noted. All patients who were consented to participate in the prospective outcomes database, underwent permanent SCS placement between Nov. 1, 2012 and Mar. 31, 2019, and had a 1-year follow-up (10-14 months) were included in our model (FIG. 1). Both demographics and pain outcome data were gathered. Pain outcomes included numeric rating scale score (NRS), PCS, MPQ, ODI, and BDI. The NRS score documents pain intensity. The PCS is a 13-item scale, with magnification, rumination, and helplessness subscales. The modified MPQ is a self-reported measure of both quality and intensity of subjective pain with affective and sensory subscores. The BDI is a self-reported measure of characteristic attitudes and symptoms of depression. The ODI, designed to assess low back pain functional outcomes, measures a patient's permanent functional disability. Pain location was also recorded.

Pain outcomes were collected in all patients pre-SCS placement and at 1-year post-operative follow-up. Patients were classified as responders if they had more than a 50% reduction of NRS (calculated as [baseline NRS-1-year NRS)/baseline NRS]×100), and as high responders if they had more than a 70% NRS reduction.

### Features

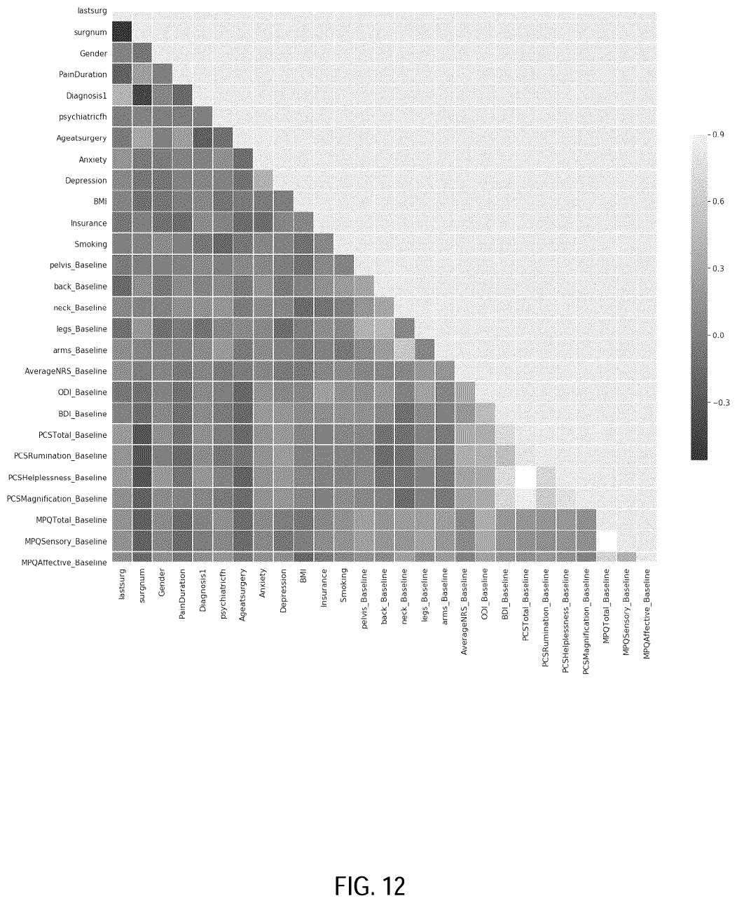

The database contained 49 features. The focus was narrowed to variables that could serve as pre-operative predictors for training ML models, thus excluding 32 factors. Age, sex, body mass index (BMI), pain diagnosis (failed back surgery syndrome (FBSS), complex regional pain syndrome (CRPS), chronic neuropathic pain or others such as occipital neuralgia, plexitis, tethered cord, combined diagnosis), chronic pain duration, number of previous spinal surgeries, time elapsed from last spine surgery (in months) when relevant, presence of anxiety, presence of depression, psychiatric family history, smoking history and insurance type were collected from medical records. Pain location, current NRS, total PCS and PCS subscores, total MPQ and MPQ subscores, BDI and ODI were considered. Anxiety and depression features were processed using ordinal integer encoding (none=0, mild=1, moderate=2, severe=3). All other categorical features (SCS indication, smoking status, insurance type and pain location) were processed using one-hot encoding (none=0, exists=1). For example, pain location was divided into 5 new binary (0/1) features: arm pain (0/1), leg pain (0/1), pelvic pain (0/1), neck pain (0/1) and back pain (0/1). Multicollinearity was evaluated, and highly correlated features (>0.7) were excluded (PCS magnification. PCS rumination. PCS helplessness and MPQ sensory subscales) (See FIG. 12). Following encoding of categorical features and exclusion of correlation, a total of 31 factors were considered during model development.

TABLE 7

| Model | Hyperparameter | Values |
|---|---|---|
| Logistic | C | Log space (−4, 4, 50) |
| Regression | Penalty | L1, L2 |
| | Solver | Newton-cg, Lbfgs, Liblinear, Sag, |
| | | Saga |
| Random | Number of | 100, 200, 250, 300, 350, 400, 450, 500, 550 |
| Forest | estimators | |
| | Max features | ‘Auto’, ‘Sqrt’, ‘Log2' |
| | Max depth | 2, 3, 4, 5, 6, 7, 8, 9 |
| | Criterion | ‘Gini’, ‘Entropy’ |
| XGBoost | Max depth | 2, 3, 4, 5, 6 |
| | Gamma | 0.1, 0.3, 0.5, 0.7, 0.9, 1, 2 |
| | ETA | 0.1, 0.2, 0.3, 0.4, 0.6, 0.7, 0.9 |
| | Reg Alpha | 0.1, 0.2, 0.3, 0.4, 0.6, 0.7, 0.9 |
| | Reg Lambda | 0.5, 0.6, 0.7, 0.8, 0.9 |

TABLE 8

| | | | RF Cluster | RF Cluster | XGB | XGB |
|---|---|---|---|---|---|---|
| | LR Cluster 1 | LR Cluster 2 | 1 | 2 | Cluster 1 | Cluster 2 |
| 1 | Months for | Psychiatric | Months for | Months for | Month from | Pain |
| | last surgery | family history | last surgery | last surgery | last surgery | duration |
| 2 | Anxiety | Legs pain | Number of | Number of | Age | Psychiatric |
| | | | previous | previous | | family |
| | | | surgeries | surgeries | | history |
| 3 | Pelvic pain | Arms pain | Psychiatric | Pain | Anxiety | Age |
| | | | family | duration | | |
| | | | history | | | |
| 4 | Baseline | Baseline BDI | Age | Psychiatric | Depression | Anxiety |
| | NRS | | | family | | |
| | | | | history | | |
| 5 | Baseline BDI | Baseline PCS | Anxiety | Age | Pelvic pain | Pelvic pain |
| | | Baseline | | | Baseline | Baseline |
| 6 | Males | MPQ | Depression | Depression | NRS | PCS |
| 7 | FBSS | Medicare | BMI | BMI | Baseline | CRPS |
| | diagnosis | | | | BDI | diagnosis |
| 8 | CRPS | No Fault | Pelvic pain | Pelvic pain | Baseline | Neuropathy |
| | diagnosis | insurance | | | PCS | indication |
| 9 | Workers | Workers | Baseline | Back pain | No Fault | Medicare |
| | compensation | Compensation | BDI | | insurance | |
| 10 | Current | Current | Current | Baseline | Current | Current |
| | smoking | smoking | smoking | PCS | smoking | smoking |
| | Parameters | Parameters | Parameters | Parameters | Parameters | Parameters |
| | C = 0.39 | C = 51.794 | Criterion = | Criterion = | ETA = 0.9 | ETA = 0.7 |
| | | | Gini | Gini | | |
| | Penalty = L2 | Penalty = L2 | Max | Max | Gamma = 2 | Gamma = 2 |
| | | | depth = 5 | depth = 3 | | |
| | Solver = | Solver = | Max | Max | Max | Max |
| | Newton-Cg | Newton-Cg | features = | features = | depth = 3 | depth = 5 |
| | | | log2 | sqrt | Alpha = 0.1 | Alpha = 0.7 |
| | | | Estimators = | Estimators = | Lambda-0.7 | Lambda = 0.6 |
| | | | 300 | 100 | | |

What is claimed is:

1. A system for predicting an outcome of neuromodulation treatment, comprising:
a user interface configured to accept data representing a plurality of features from a new patient for whom a prediction of spinal cord stimulation is desired; and
a machine learning engine having a cluster stage trained to evaluate the plurality of patient features to identify a cluster corresponding to the plurality of features of the patient from a plurality of clusters and a prediction stage trained to output a patient predicted outcome based a predictive model corresponding to the identified cluster.

2. The system of claim 1, wherein the plurality of clusters of the cluster stage are defined according to K-means clustering of data representing the plurality of features from patients having known outcomes.

3. The system of claim 2, wherein the predictive model comprises a machine learning algorithm trained with data representing the plurality of features from patients having known outcomes.

4. The system of claim 3, wherein the machine learning algorithm is selected from the group consisting of logistic regression, random forest, XGBoost, elasticnet, support vector machine, Naïve Bayes, and combinations thereof.

5. The system of claim 4, wherein the plurality of features are selected from at least one of demographics, pain descriptors, pain questionnaire data, psychiatric comorbidities, spinal imaging, activity, medications, non-psychiatric comorbidities, and past spinal cord stimulation results.

6. A method for predicting an outcome of neuromodulation treatment, comprising:
collecting a plurality of patient features from a patient whose spinal cord stimulation outcome is to be predicted;
using a machine learning engine having a cluster stage trained to evaluate the plurality of patient features to identify a cluster corresponding to the plurality of features of the patient from a plurality of clusters; and
using the identified cluster to evaluate the patient data with a prediction stage of the machine learning engine trained to output a patient predicted outcome based a predictive model that corresponds to the identified cluster.

7. The method of claim 6, wherein the plurality of clusters of the cluster stage are defined according to K-means clustering of data representing the plurality of features from patients having known outcomes.

8. The method of claim 7, wherein the predictive model comprises a machine learning algorithm trained with data representing the plurality of features from patients having known outcomes.

9. The method of claim 8, wherein the machine learning algorithm is selected from the group consisting of logistic regression, random forest, XGBoost, elasticnet, support vector machine, Naïve Bayes, and combinations thereof.

10. The method of claim 9, wherein the plurality of features are selected from at least one of demographics, pain descriptors, pain questionnaire data, psychiatric comorbidities, spinal imaging, activity, medications, non-psychiatric comorbidities, and past spinal cord stimulation results.
